(19) United States
(12) Patent Application Publication
(10) Pub. No.: US 2026/0041891 A1
(43) Pub. Date: Feb. 12, 2026

(54) MEDICAL ACCESS SYSTEM

(71) Applicant: EBNET MEDICAL GMBH, Schwerin (DE)

(72) Inventors: Jens EBNET, SCHWERIN (DE); Werner SCHWARTZ, RUHPOLDING (DE)

(21) Appl. No.: 19/101,086

(22) PCT Filed: Aug. 16, 2023
(86) PCT No.: PCT/EP2023/072555

§ 371 Date: Feb. 4, 2025

(30) Foreign Application Priority Data
Aug. 19, 2022 (DE) 10 2022 121 022.7

(51) Int. Cl. A61M 25/06; A61M 25/00

(52) U.S. Cl. CPC A61M 25/0606; A61M 25/0045; A61M 2025/0046; A61M 2025/0059

(57) ABSTRACT

The invention relates to a medical access system for providing an access to a hollow body of a patient, wherein the access system has at least one catheter with a vasiform catheter tube, wherein the catheter is designed to be pushed over at least a part of the length of the catheter tube through an opening created by means of a puncture needle or in another way through a tunic of the hollow body after puncture of the tunic of the hollow body to be punctured has been carried out, and to remain there over a period of time. Such a hollow body of a patient may for example be a pipe-shaped vessel, in particular a blood vessel, for example a vein or artery, or a lymphatic vessel, or another hollow organ.

### FIELD OF THE INVENTION

The invention relates to a medical access system for providing an access to a hollow body of a patient, wherein the access system has at least one catheter with a vasiform catheter tube, wherein the catheter is designed to be pushed over at least a part of the length of the catheter tube through an opening created by means of a puncture needle or in another way through a tunic of the hollow body after puncture of the tunic of the hollow body to be punctured has been carried out, and to remain there over a period of time. Such a hollow body of a patient may for example be a pipe-shaped vessel, in particular a blood vessel, for example a vein or artery, or a lymphatic vessel, or another hollow organ.

### BACKGROUND

Medical access systems are known in various forms, for example as an indwelling cannula or venous catheter, for example for dialysis. Such an indwelling cannula may, for example, be configured as an indwelling venous cannula. The medical access system may, for example, be configured as a puncture system/catheter system.

The terms “puncturing”and “puncture”are to be understood in the medical sense. “Puncturing” refers to the entry of a puncture needle into the hollow body in such a way that the puncture needle penetrates through the tunic of the hollow body to be punctured.

Those parts which are arranged at the remote (distal) end of the indwelling cannula from the viewpoint of the user and therefore in the vicinity of the punctured hollow body, are regarded as a section of parts of the indwelling cannula near to the hollow body. Correspondingly, sections remote from the hollow body are arranged at the proximal end of the indwelling cannula from the user's viewpoint, i.e. further away from the punctured hollow body. In connection with medical applications of the indwelling cannula, the terms “near-vein” or “near-patient” or “user-remote” are also used synonymously with the term “near to the hollow body”, and “vein-remote” and “patient-remote” and “near-user” are also used for the term “remote from the hollow body”.

Near-vein components tend to be located inside the patient, and vein-remote components tend to be located outside the patient. This need not necessarily always apply, but is meant to further clarify the terms. The additions are regularly self-evident, and the context is crucial.

A piercing-resistant indwelling venous cannula is disclosed by PCT/EP2019/057097.

### SUMMARY

An improved medical access system in the sense of a general puncture system/catheter system is intended to be provided. In principle, with the improved medical access system it is likewise possible advantageously to puncture all body cavities and interstitial body spaces and all anatomical and morbid structures that are intended to be punctured, and to install a catheter in them. Only puncture, without installation of a catheter, may also take place.

Components of the improved medical access system may in principle also be combined with all known puncture and catheter systems or else be used as independent products.

The term “indwelling cannula” will sometimes be adopted below, although it has a wide meaning in the sense of a general medical access system or puncture system with which not only veins may be punctured. In what follows, the terms “vein” and “veins” thus include in principle all blood vessels and very generally all body cavities and interstitial body spaces and all anatomical and morbid structures that are intended to be punctured and provided with a catheter.

Thus, in addition, by way of example a trachea, pleural space, abdomen, stomach, intestine, renal pelvis, urinary bladder, structures of the central and peripheral nervous system, subarachnoid space and bone may be punctured with the indwelling cannula. In addition, morbid structures such as abscesses in and on the patient may be punctured. Advantageously, arterial blood vessels may also be punctured.

In what follows, the term “patient” includes all living beings of all age groups and genders. Applications in technical fields and in and on all objects and structures are likewise explicitly possible, for example in and on reservoirs, containers, cavities, extensible materials and in and on pump, hose, pipe and port systems.

When the word “or” is used in what follows, possible alternatives are indicated but combinations of the features or configurations separated by “or” are in principle also explicitly possible.

All components described may be employed singly or multiply on an indwelling cannula or also, independently of such an indwelling cannula, on/in other products or entirely independently. Various features of different components are also freely combinable, and features of particular components may also be used on other components without this being explicitly mentioned. All components and features may in principle be employed inside and outside a patient.

Indwelling cannulas, and in particular indwelling venous cannulas, are special medical devices insofar as they must have particular diameter and length proportions in order to be able to be applied in human or animal patients at the usual access sites on veins. For this purpose, a certain flexibility or resilience of the catheter tube is also necessary. Such indwelling cannulas are therefore not comparable with catheter systems for other applications, for example a urinary catheter, because entirely different requirements apply in those cases.

The object of the invention is to provide a medical access system which is easier and safer to use.

This object is achieved by a medical access system as claimed in claim 1. Advantageous developments are specified in the dependent claims.

According to the invention, it is provided that at least the part of the catheter tube which is designed to remain in the hollow body has at least one spiral supporting structure, particularly in the form of at least one spirally wound, internally hollow guide wire, over its entire length or over the predominant part of its length. Because of the spiral shape of the supporting structure, an inner cavity through which the puncture needle can be guided is formed therein. The catheter tube can be supported by the spiral supporting structure arranged in the catheter tube, and in particular sharp kinking of the catheter tube can be avoided. The inner lumen of the catheter tube is kept permanently open by the spiral supporting structure, and no obstructions therefore occur. The bending moment of resistance and/or the radial moment of resistance of the catheter tube is increased by the spiral supporting structure.

Because of the spiral supporting structure, the catheter tube can be adapted to the profile as well as branchings of for example blood vessels or other anatomical structures without sharp kinking. Besides use in/on indwelling cannulas, this may also be particularly advantageous when the catheter tube according to the invention is configured to be relatively long, for example up to 200 cm, and needs to be pushed forward over a relatively large distance into the body, for example in applications in the field of interventional cardiology or (neuro)radiology or in the scope of minimally invasive surgical interventions.

Any conventional/commercially available, single-lumen or multilumen, catheter tube or other hose with all diameter and length proportions may be used.

The supporting structure may, for example, be a spiral structure extending over a certain length section of the catheter tube. There may also be a plurality of coaxial supporting structures of this type. The spiral supporting structure may also have spring-like properties. The spiral structure may be formed from round material or flat material, in particular from a metal material. The catheter tube may be configured in a single layer or multiple layers in the radial direction, both from the same and from different materials in the individual radial layers.

The supporting structure may be configured to be undulating or have an undulating surface. An undulating surface is characterized at least in cross section along the longitudinal axis of the supporting structure by alternating diameters of the supporting structure. A waveform may, for example, consist of a sinusoidal, rectangular, triangular and/or sawtooth oscillation. The supporting structure may also consist of at least one braid-like structure or be developed with at least one such structure. The supporting structure may also consist of at least one structure running longitudinally, transversely or diagonally with respect to the longitudinal axis of the catheter tube or be developed with at least one such structure. At least one such structure may also cover or bridge a certain length of the supporting structure and therefore oppose a length change, for example, in the event of a spiral supporting structure uncoiling.

Other configurations and shapes of the supporting structure and its surface are also conceivable. For instance, the supporting structure may have both a smooth and a pimpled surface. The surface of the supporting structure may consist of at least one material or be coated with at least one material, which is distinguished by an increased or high slideability.

For example, polyurethane (PU) or FEP are envisioned as materials for the catheter tube. Materials for the supporting structure may be metal materials, in particular stainless steel and/or metal alloys, in particular nitinol. Nitinol has the advantage that the desired resistance of the catheter tube against compression or kinking is increased further by the memory effect.

Further materials for the supporting structure may be plastic materials, in particular also fiber-reinforced, for example carbon or aramid fiber-reinforced, plastics. The materials for the supporting structure may be polymers. The materials for the supporting structure may be polycondensates. The materials for the supporting structure may be polyadducts. The materials for the supporting structure may be thermosets or thermoplastics. Plastic materials that are distinguished by high mechanical strength, for example polyimides, may also be used. Plastic materials that have rubber-elastic properties, for example silicone elastomers, may also be used. Resins may also be used, for example unsaturated polyester resins. Polyethylene may also be used. Polytetrafluoroethylene (PTFE) may also be used. Polyvinyl chloride (PVC) may also be used. For instance, the supporting structure may be configured as a hard-PVC spiral, for example.

Advantageously, such materials that have a sufficient or good magnetic resonance tomography (MRT) capability may in principle be used as materials for the supporting structure. Advantageously, such materials that have a sufficient or good insulating power against heat and/or electric current may in principle be used as materials for the supporting structure. Such materials may also be put into the spiral supporting structure with interruptions by other materials. Also, only particular locations of the spiral supporting structure may be configured as locations with sufficient or good insulating power. It is also conceivable that special adhesives are used for this.

The catheter tube may consist of one or more of the aforementioned materials. The supporting structure may consist of one or more of the aforementioned materials.

Advantageously, compound materials and such materials that expand or become more solid when heated by the body temperature may also be employed. The spiral supporting structure may fully or partly be a bimetal.

The supporting structure may also partially, predominantly or fully be formed from at least one material that is resorbable/readily soluble in blood and infusion solutions or be coated with such a material. This resorbable material may be a carbohydrate, or alternatively another biomolecule or a salt. This material may also be or contain magnesium. Resorbable polymers, composites, bioceramic materials or biodegradable metals may likewise be employed. The material may also be a combination of a plurality of resorbable materials and may also contain at least one antimicrobial or anticoagulant or otherwise effective or biologically active substance or be coated with at least one such substance.

The supporting structure may also be used as, or be part of, a sensor and/or detector in order to detect particular properties or state changes of the catheter tube, for example compression or bending of the catheter tube. Advantageously, properties or state changes in the region of the catheter tube may also be detected by such a sensor and/or detector, for example in respect of the following parameters/measurement values of the blood: temperature, oxygen content, electrolytes, pH, blood sugar, renal and hepatic function. In the event of a shape/state change of the supporting structure which is pulse-synchronous and/or associated with blood pressure level, i.e. an expansion in the radial or longitudinal direction, in the case of arterial placement of the catheter tube according to the invention it is also conceivable to measure parameters that are used to determine the arterial blood pressure and values associated therewith, in order to be able to assess the circulatory system function of the patient. In the case of venous placement of the catheter tube according to the invention, it is conceivable to measure the venous pressure. It is also conceivable that the widths of blood vessels are measured by the supporting structure, in particular also a change in the tone (state of tension) of blood vessels.

In the case of a spiral configuration of the supporting structure, there may be one spiral or a plurality of spirals placed in one another. Of such spirals, at least one may also be configured as a sensor and/or detector. When there are a plurality of spirals, they may consist of different materials. Advantageously, at least one spiral may be electrically conductive and at least one further spiral may be electrically insulating. The turns of the spirals may have the same spacing from one another over the entire length of the supporting structure or varying spacings. In one advantageous configuration of the invention, a spiral supporting structure may have a smaller spacing of the turns from one another in the region remote from the hollow body than in the region near to the hollow body.

By such a supporting structure, when using a material that is visible in imaging medical methods, in particular a metal material, the visibility of the catheter in patients by means of imaging medical methods, for example by ultrasound scanning, may be improved.

According to one advantageous configuration of the invention, it is provided that the spiral supporting structure is arranged over the predominant part of its longitudinal extent or over its entire longitudinal extent loosely in the catheter tube. The supporting effect of the spiral supporting structure may be further optimized in this way. In the event of the catheter tube bending, the spiral supporting structure may loosely follow this shape change without excessive stresses occurring in the material of the spiral supporting structure. In particular, an improvement of the kink resistance may thereby be achieved in comparison with embodiments in which the spiral supporting structure is embedded in the material of the catheter tube or in another material and the individual spiral turns cannot therefore move freely in relation to the catheter tube.

The spiral supporting structure may, for example, bear loosely on the inner side of the catheter tube. The spiral supporting structure may execute a slight relative movement in relation to the catheter tube in the event of the catheter tube bending. Even if it is fastened to the catheter tube or another part of the indwelling cannula, the spiral supporting structure is therefore at least slightly displaceable relative to the catheter tube in the unfastened regions. The spiral supporting structure may have a certain play in the radial direction in relation to the inner side of the catheter tube.

It is also conceivable that the catheter tube also contains sections over the length of which a spiral supporting structure is present, albeit one which is arranged not loosely but fixed in the catheter tube. It is also conceivable that the extent of the relative movement of the spiral supporting structure in relation to the catheter tube is different in various sections of the catheter tube.

The spiral supporting structure may, for example, be configured as a portion of a Seldinger wire. In contrast to a Seldinger wire, however, the spiral supporting structure is secured to the catheter tube so that the spiral supporting structure cannot fall out or emerge from the catheter tube due to improper use. Once the catheter tube is placed on the patient, the spiral supporting structure remains in the catheter tube throughout the entire installation time.

According to one advantageous configuration of the invention, it is provided that the spiral supporting structure is secured using at least one fastening element permanently to the catheter tube or a constituent part of the medical access system, which is connected thereto. Such a fastening element ensures that the spiral supporting structure does not fall out of the catheter tube or change its position to an undesired extent. The individual turns of the spiral supporting structure, however, are still arranged predominantly loosely in the catheter tube. In contrast to known applications with a Seldinger wire, the spiral supporting structure therefore differs by its permanent arrangement inside the catheter tube, without protruding therefrom and without being removable therefrom.

According to one advantageous configuration of the invention, it is provided that the spiral supporting structure is secured at one of its ends or at both ends using at least one fastening element to the catheter tube or a constituent part of the medical access system, which is connected thereto, and is otherwise arranged loosely in the catheter tube. Only end-side securing of the spiral supporting structure therefore takes place, so that the regions of the spiral supporting structure located between the ends, or in the case of fastening only on one side, also an end side remote therefrom, is/are arranged loosely in the catheter tube.

The spiral supporting structure may, for example, be secured only at one end in such a way that the spiral supporting structure is mobile neither in one axial direction nor in the opposite axial direction at this secured end. If the spiral supporting structure is secured at both ends, the spiral supporting structure may for example be inserted between two fastening elements that secure it in mutually opposite axial directions. The spiral supporting structure may then have a certain axial play between the two fastening elements, i.e. it may be slightly displaceable in the axial direction.

According to one advantageous configuration of the invention, it is provided that a tubular inner body is arranged inside the spiral supporting structure. In this way, the spiral supporting structure is shielded inward by the tubular inner body, for example against a puncture needle being guided through there or against liquids being conveyed. The spiral supporting structure may be arranged with a certain radial play on the tubular inner body. The tubular inner body extends in the longitudinal direction through the spiral structure. The tubular inner body may extend over the entire length of the spiral supporting structure or only over a part of this length. The tubular inner body may also be longer than the spiral supporting structure and protrude therefrom on one or both end sides.

For example, the catheter tube may thus have a tubular outer body and a tubular inner body, which is arranged with a radial spacing inside the tubular outer body, an annular gap in which the spiral supporting structure is arranged being formed between the tubular outer body and the tubular inner body. The spiral supporting structure may be arranged with a certain radial play in the annular gap. There may additionally be a substance in the annular gap, for example a substance containing oil or fat or another substance or liquid, which reduces the frictional resistance between the spiral supporting structure and its surroundings. The displaceability according to the invention of the spiral supporting structure in relation to its surroundings is therefore further improved. Such a substance or liquid may for example also have other properties, which for example keep the spiral supporting structure and its surroundings in a particular temperature range or protect the material used against modification, wear or destruction, for example fracture or corrosion, or against infection with germs.

The tubular inner body may also be assigned in and/or on a module of the medical access system other than the catheter tube, for example the supporting module further explained below. The tubular inner body may, for example, be configured as a pipe or a hose.

In an alternative configuration, the spiral supporting structure is not covered inward with an inner layer, i.e. it is exposed and may for example come into contact with liquids conveyed through the catheter tube. If a puncture needle is guided through the catheter tube, it may for example bear directly on the inner side of the spiral supporting structure.

According to one advantageous configuration of the invention, it is provided that the spiral supporting structure is secured releasably to the catheter tube or a constituent part of the medical access system, which is connected thereto, and if required can be withdrawn from the catheter tube. This has the advantage that the spiral supporting structure can be removed or replaced even when the catheter is already applied on the patient. This may, for example, be expedient if obstruction occurs in the catheter tube. After removal of a spiral supporting structure from the catheter tube, it is also possible to insert another spiral supporting structure there, which has the same length or a different length, for example a spiral supporting structure which is configured to be longer overall than the spiral supporting structure originally arranged there. There may also be a plurality of removable or replaceable spiral supporting structures.

According to one advantageous configuration of the invention, it is provided that the medical access system has a supporting module as a further module, which is present in addition to the catheter and is coupled releasably to the catheter, for example to a housing of the catheter, the supporting module having the spiral supporting structure. This has the advantage that the spiral supporting structure can be removed by the user straightforwardly from the catheter tube by the entire supporting module being removed. For example, the user may grip a housing of the supporting module in order to remove the supporting module. In the production and delivery state of the medical access system, said system may then be delivered with all the modules, i.e. at least with the catheter and the supporting module fastened thereto. Depending on the configuration of the medical access system, the needle device may additionally be arranged thereon.

According to one advantageous configuration of the invention, it is provided that the spiral supporting structure is secured permanently to a part of the supporting module, for example to a housing of the supporting module. For example, the spiral supporting structure may be molded into the material of a part of the supporting module or permanently fastened thereto by a form fit or in another way, for example by adhesive, welding and/or plugging.

In one advantageous configuration of the invention, the housing of the supporting module may have fastening elements that are assigned to corresponding fastening elements of the housing of the catheter, which are configured as a counterpart thereto. Using these fastening elements, the supporting module may then be fastened to the catheter by the housing of the supporting module being coupled to the housing of the catheter. For example, the fastening elements may be configured as a bayonet connection and/or as latching elements. There may also be a plurality of supporting modules.

When replacing the spiral supporting structure, after removal of a spiral supporting structure, another spiral supporting structure may be inserted, for example a spiral supporting structure that has the aforementioned tubular inner body. In this case, the tubular inner body may protrude at the near-patient end beyond the spiral supporting structure to such an extent that, when the spiral supporting structure is guided fully into the catheter tube, the tubular inner body protrudes from the near-patient end of the catheter tube and is therefore guided for example into a vein or another hollow body of the patient. With such a tubular section protruding from the catheter tube at the near-patient end, a venous valve may for example be bridged.

Alternatively, such a tubular section protruding from the catheter tube at the near-patient end may also be provided by an additional constituent part which is applied at the near-patient end of the spiral supporting structure, for example by welding, plugging and/or adhesive bonding. In this case, the spiral supporting structure may also be configured without the tubular inner body. The aforementioned tubular section configured as an additional constituent part is then applied to the near-patient end of the spiral supporting structure.

According to one advantageous configuration of the invention, it is provided that the spiral supporting structure has a multiplicity of spiral turns, which bear directly on one another. This leads to a good supporting effect of the spiral supporting structure. The spiral supporting structure may have spiral turns that lie directly on one another only in one or more individual regions; it may also be configured in this way over its entire length. The spiral supporting structure is therefore wound tightly in these regions, for example in a similar way to a Seldinger wire. There is no intermediate space between the individual turns. The region of the spiral turns bearing directly on one another may extend over the predominant part of the longitudinal extent of the spiral supporting structure or over the entire longitudinal extent. The spiral supporting structure may also have one or more regions in which the spiral turns are spaced apart from one another, in which case the spiral turns may have a pitch that is constant over the length or a variable pitch in these regions.

According to one advantageous configuration of the invention, it is provided that the spiral supporting structure has a multiplicity of spiral turns, which loosely bear directly on one another but are not fastened to one another, for example not adhesively bonded or the like, in particular not embedded into a connecting material. In this way, the spiral turns can be displaced in relation to one another when the catheter tube is bent. Stresses on the spiral supporting structure are thereby optimally dissipated so that the supporting effect is further improved.

According to one advantageous configuration of the invention, it is provided that the spiral supporting structure or its spiral turns are provided with a friction-reducing coating, in particular a PTFE coating. For example, this facilitates the extraction of a puncture needle from the spiral supporting structure. Alternatively or in addition, the inner side of the catheter tube may also be provided with such a friction-reducing coating, in particular a PTFE coating.

According to one advantageous configuration of the invention, it is provided that the spiral supporting structure extends only over a part of the longitudinal extent of the catheter tube. The spiral supporting structure is therefore shorter than the catheter tube and thus cannot protrude from the catheter tube, in particular not from the near-patient end, in any operating state of the access system. The length indication refers to the length of the spiral supporting structure in the wound state, not in the stretched state of the material of the spiral supporting structure.

The diameter of the spiral supporting structure may be constant over its entire length. Advantageously, the inner diameter of the spiral supporting structure may be selected in such a way that a puncture needle can be guided with little play through the spiral supporting structure.

According to one advantageous configuration of the invention, it is provided that the catheter tube ends at the near-patient end with an axial opening, the inner diameter of which is less than the outer diameter of the spiral supporting structure or at least of the near-patient end region of the spiral supporting structure. The axial opening is used, for example, as an outlet opening of the catheter tube for the administration of medications to the patient. When a puncture needle is guided through the catheter tube, the axial opening is thus used to guide the puncture tip of the puncture needle out of the near-patient end of the catheter tube, for example in order to carry out a puncture.

According to one advantageous configuration of the invention, it is provided that the medical access system is configured as an indwelling cannula for puncturing a hollow body of a patient by means of a puncture needle, wherein the indwelling cannula has at least the catheter with the vasiform catheter tube, in which the puncture needle can be guided longitudinally displaceably, wherein the catheter is designed to be pushed over at least a part of the length of the catheter tube through the opening created by means of the puncture needle through a tunic of the hollow body to be punctured after puncture of the tunic of the hollow body to be punctured has been carried out, and to remain there over a period of time. This ensures that the spiral supporting structure cannot protrude from the near-patient end of the catheter tube under any circumstances, even in the event of incorrect operation of the access system.

Such an indwelling cannula may for example be configured as an indwelling venous cannula, also referred to as a peripheral venous catheter.

Such an indwelling cannula may also be configured as an arterial catheter.

Such an indwelling cannula may also be configured as a catheter for puncturing the pleural space.

The catheter tube according to the invention with the spiral supporting structure may also be used on/with medical access systems that are used for puncturing central veins. The catheter tube with the spiral supporting structure may thus be part of central venous catheter systems. In one advantageous configuration of a central venous catheter system, a plurality or all of the hoses (lumens, branches) leading into the vein and branching off from the mains hose are configured as an aforementioned catheter tube according to the invention with the spiral supporting structure. It is however also conceivable that only selected lumens are configured according to the invention, for example those that need to be especially safeguarded against kinking. Selected lumens may also be configured only in sections over at least a partial length or over the predominant length as a catheter tube according to the invention with the spiral supporting structure. It is also possible that only the region of the lumens where the lumens enter through the skin of the patient and/or into the vein are configured in the manner of the catheter tube according to the invention, since the risk of the catheter tube kinking is particularly high in this region. The catheter tube according to the invention with the spiral supporting structure may also be used on/with pulmonary artery catheters.

The catheter tube according to the invention with the spiral supporting structure may also be used on/with medical puncture/catheter systems (access systems) in the field of interventional cardiology and interventional radiology and neuroradiology.

The catheter tube according to the invention with the spiral supporting structure may also be used independently, without being a part of a medical puncture/catheter system. For example, it may thus be used on/with an infusion/transfusion hose or may itself constitute one. Such a hose may also be configured so that it is suitable for high flow rates, which for example allow dialysis to be carried out. The catheter tube according to the invention may have a length of up to 10 meters, preferentially a length of from 1 to 2 meters. The spiral supporting structure is then also correspondingly lengthened. It is also conceivable that the spiral supporting structure covers only the regions of the catheter tube that need to be especially protected against kinking, that is to say the spiral supporting structure is not arranged in the entire catheter tube. In such a case, the spiral supporting structure may also be connected at a plurality of locations to the catheter tube or the catheter tube may contain a plurality of spiral supporting structures delimited from one another. If external mechanical deformation of the catheter tube is desired, for example when using a hose pump or peristaltic pump, at the corresponding location there may be no spiral supporting structure or a spiral supporting structure with special properties. It is also conceivable that the catheter tube also contains sections over the length of which there is a spiral supporting structure, albeit one which is arranged not loosely but fixed in the catheter tube.

The catheter tube according to the invention with the spiral supporting structure may also be a component part of an infusion/transfusion system.

The catheter tube according to the invention with the spiral supporting structure may also be a component part of a puncture/catheter system for carrying out regional anesthesiological and interventional pain management methods. This includes both peripheral methods and near-spine and near-joint methods.

The catheter tube according to the invention with the spiral supporting structure may also be a component part of a puncture/catheter system for discharging cerebrospinal fluid.

The catheter tube according to the invention with the spiral supporting structure may also be a component part of a puncture/catheter system for discharging urine.

The catheter tube according to the invention with the spiral supporting structure may also be a component part of a drainage system which discharges fluids, for example ichor, from the body.

The catheter tube according to the invention with the spiral supporting structure may also be used independently in order to be introduced into blood vessels and keep them open over a prolonged period of time and/or also for example to cover/bridge defects on the blood vessel wall. For instance, the catheter tube according to the invention with the spiral supporting structure may be used as a stent, for example in the coronary vessels. It may also be used as a stent or vascular prosthesis in larger blood vessels, for example in arm and leg vessels and in the aorta or the vena cavae (vena cava inferior/superior). For this purpose, sometimes considerable differences of the diameter and length dimensions from those of, for example, indwelling cannulas upward are conceivable, for example up to 4 cm in diameter and up to 120 cm in length.

The catheter tube according to the invention with the spiral supporting structure may also be used as a dialysis shunt or as an element thereof.

The catheter tube according to the invention with the spiral supporting structure may also be used as a hose which is not necessarily intended for medical use. For instance, it may also be used as a hose in the field of sanitation, for example as a shower hose, with the diameter and length dimensions of conventional there. Use as a garden hose is also conceivable, likewise use as fuel hose or fire hose.

It is in principle conceivable to use the catheter tube according to the invention with the spiral supporting structure as a hose in all fields of application in which sharp kinking of hoses should/must be prevented. The catheter tube according to the invention with the spiral supporting structure may therefore also be used as an independent hose, which may be employed in a multitude of fields of application.

The catheter tube according to the invention with the spiral supporting structure may also be at least one component of a multitude of devices, machines, pump systems, installations and for example also motors and means of transport/locomotion.

In all further configurations of the catheter tube according to the invention which are mentioned above, significant differences upward or downward in respect of diameter and length dimensions as well as wall thickness in comparison with indwelling cannulas are in principle conceivable and partly necessary. In the downward direction, this may also include the nanoscale (nanostructures).

The catheter tube according to the invention with the spiral supporting structure may, in particular, be bent and/or angled off at at least one bending or angular location in the region of the vein-remote transition into further components of a medical access system, for example of an indwelling venous cannula. When the indwelling venous cannula lies in the patient, the bending or angular location of the catheter tube according to the invention with the spiral supporting structure is located for example the region of the skin level. There, a catheter tube may be exposed to particular mechanical loads because of the way it is used. In contrast to a catheter tube running straight from the indwelling venous cannula in the direction of the vein, the catheter tube according to the invention with the spiral supporting structure may be bent and/or angled off at the bending or angular location by at least 1° in relation to a straight line, but preferentially at an angular measurement of from 2 to 5°, 6 to 10°, 11 to 25°, 26 to 45°or more than 45°. The catheter tube according to the invention with the spiral supporting structure therefore forms a bend, which may be directed upward or downward. A puncture needle guided through the catheter tube according to the invention with the spiral supporting structure may also be configured to be bent and/or angled off as just described. This may facilitate puncture processes since, for example, puncturing can be carried out more shallowly or even more individually matched to the anatomical situations in the region of bony prominences or joints.

The catheter tube according to the invention with the spiral supporting structure may be developed with at least one further network-, braid-or grid-like structure in the region of the skin level or else in other mechanically overly stressed regions. A combination with transversely, longitudinally or diagonally running structures is conceivable, and these may ensure a desired/increased stability of the wall of the catheter tube at least in sections on their own, without a spiral supporting structure needing to be present at least in sections. The catheter tube may also be developed in the manner just described or in a further manner in other critical regions, for example in the region of the entry site into the vein or in the region of its distal end.

If a puncture needle guided through the catheter tube according to the invention with the spiral supporting structure has a tip with a bevel, various puncture techniques are possible. In the bevel-up technique, the puncture needle tip is guided through the skin in such a way that the bevel, including the inner opening of the puncture needle that is present therein, points upward, i.e. it is turned away from the skin of the patient. In the bevel-down technique, the puncture needle tip is rotated by 180° in comparison therewith in relation to the catheter tube, so that the bevel, including the inner opening of the puncture needle that is present therein, points downward, i.e. it is turned toward the skin of the patient.

The medical access system according to the invention, for example the indwelling venous cannula, may for example be configured in such a way that the puncture needle, or at least the puncture needle tip, can at any time be twisted about the longitudinal axis in relation to the catheter tube according to the invention with the spiral supporting structure. The user may then selectively employ the bevel-up technique or the bevel-down technique. The catheter tube according to the invention with the spiral supporting structure can facilitate this twisting about the longitudinal axis since the configuration according to the invention of the catheter tube with the spiral supporting structure prevents sharp kinking thereof, which might make it difficult to twist the puncture needle about the longitudinal direction.

The medical access system according to the invention, for example the indwelling venous cannula, may also be configured in such a way that in at least one particular longitudinal displacement position, for example when the puncture needle tip protrudes at the near-patient end from the catheter tube according to the invention with the spiral supporting structure, the puncture needle or at least the puncture needle tip cannot be twisted in relation to the catheter tube according to the invention with the spiral supporting structure, or can be twisted only with increased force exertion. For example, a module having the puncture needle may be coupled in this longitudinal displacement position with a form fit to a module having the catheter tube according to the invention with the spiral supporting structure, and in this way the twistability of the puncture needle in relation to the catheter tube according to the invention with the spiral supporting structure may be prevented.

The medical access system according to the invention, for example the indwelling venous cannula, may for example be configured in such a way that the bevel, including the inner opening of the puncture needle that is present therein, points downward from the start, i.e. it is turned toward the skin of the patient or a lower side of the indwelling cannula, on which at least one fixing wing of the indwelling cannula is arranged.

According to a further advantageous configuration of the medical access system according to the invention, for example the indwelling venous cannula, it is provided that the puncture needle tip is twisted/rotated about its own longitudinal axis and/or the longitudinal axis of the puncture needle, and can preferentially be set to a circular measurement of 45°, 90°, 135°, 180°, 225°, 270°, 315°or 360°(angle specifications in degrees with respect to a circular measurement of 360 degrees (360°)), all intermediate stages being conceivable in respect of the circular measurement.

Because of the design, in this case, the bevel of the puncture needle tip already no longer necessarily points upward before use of the medical access system according to the invention, for example the indwelling venous cannula, and is thus no longer necessarily turned away from the skin of the patient. This may allow advantageous gentler puncture techniques since, inter alia, puncture/entry angles can be varied.

The puncture needle or a module having the puncture needle may have holding/path-limiting elements (in what follows, only “holding elements”) by which the twistability/rotatability of the puncture needle tip/puncture needle in relation to the catheter tube according to the invention with the spiral supporting structure is restricted or negated, at least in particular longitudinal displacement positions of the puncture needle tip and/or puncture needle and/or needle device.

In one advantageous configuration of the medical access system according to the invention, for example the indwelling venous cannula, the twistability/rotatability just described of the puncture needle tip/puncture needle is fully negated when the puncture needle tip projects distally (in the direction of the patient) at least partially beyond the catheter tube according to the invention with the spiral supporting structure. The holding elements may, for example, be configured as latching elements, notches, furrows or projections. These may interact with holding elements that are located on the catheter tube according to the invention with the spiral supporting structure or on further components of the medical access system according to the invention, for example the indwelling venous cannula, for example by latching, wedging or twisting. All holding elements may at least partially consist of a material or be coated with at least one such material that increases the frictional resistance. The aforementioned angle specifications may be made discernible for the user on a component of the medical access system, for example the indwelling venous cannula, so that the current circular measurement at which the puncture needle tip and/or puncture needle is set can be read off easily. Here, for example, circular or semicircular markings in a different line width and, for example, also colored markings in the traffic light colors “green”, “yellow” and “red” are conceivable too. It is also conceivable that initial puncture and further pushing of the puncture needle tip and/or puncture needle forward into the tissue to be punctured are carried out in such a way that the puncture needle tip and/or puncture needle is sequentially set to a different circular measurement, i.e. twisted/rotated about the longitudinal axis of the puncture tip/puncture needle in the chronological sequence of the puncture process.

In one further advantageous embodiment, the invention relates to a medical access system for providing an access to a hollow body of a patient, wherein the access system has at least one catheter with a vasiform catheter tube, wherein the catheter is designed to be pushed over at least a part of the length of the catheter tube through an opening created by means of a puncture needle or in another way through a tunic of the hollow body after puncture of the tunic of the hollow body to be punctured has been carried out, and to remain there over a period of time, wherein at least the part of the catheter tube which is designed to remain in the hollow body has at least one supporting structure over its entire length or over the predominant part of its length. Said supporting structure need not be configured in the form of a spiral, but may for example be configured in the form of a cylinder, pipe or hose. Such a supporting structure may advantageously be developed with the features explained above and below for the spiral supporting structure. For example, parts of the catheter tube may be supported by such a supporting structure. There may also be a plurality of supporting structures which are configured in this way and are arranged displaceably in relation to the puncture needle, the catheter tube and/or one another, in particular also longitudinally displaceably, also for example in the sense of a telescopic arrangement.

### DESCRIPTION OF THE DRAWINGS

The invention is explained in more detail below with the aid of exemplary embodiments with the use of drawings, in which

FIG. 1 shows a medical access system in the form of an indwelling cannula in a perspective view,

FIG. 2 shows the near-patient region of a catheter tube in a side view,

FIG. 3-5 show the near-patient region of a catheter tube in a side sectional representation in various embodiments,

FIG. 6 shows a cross-sectional view of the catheter tube according to FIG. 5,

FIG. 7 shows a further embodiment of a medical access system in a perspective view,

FIG. 8 shows the system according to FIG. 7 in an exploded representation,

FIG. 9 shows parts of the system according to FIG. 7,

FIG. 10 shows a supporting module,

FIG. 11 shows the supporting module according to FIG. 10 with a catheter.

### DESCRIPTION

The medical access system represented in FIG. 1 has a catheter 29 and a needle device 39. The needle device 39 forms a separate structural unit from the catheter 29, which can be separated from the catheter 29 after a puncture has been carried out. Only the catheter 29 then remains on the patient.

The catheter 29 has a housing 20 on which, on one side turned toward the patient, there is a tube exit region 21 at which a catheter tube 1 emerges from the housing 20 and projects therefrom. The catheter tube 1 is configured to be comparatively flexible and is used as an application option for the intravenous administration of liquids, in particular infusion solutions, blood products and medications. The catheter tube 1 is then located with its distal part in a hollow body, for example in the vein of a patient.

The needle device 39 has a puncture needle 30, which in the basic state of the medical access system is located substantially in the housing 20 and the catheter tube 1, the tip 31 of the puncture needle 30 protruding from the distal end of the catheter tube 1. The needle device 39 is displaceable in relation to the catheter 29 in the longitudinal direction L.

Arranged on the housing 20, there are fixing wings 23 which are used for manual handling and securing of the catheter 29 on the patient. On the side turned away from the tube exit region 21, the housing 20 has a needle opening 25 through which the puncture needle 30 can be placed in the housing 20 and in the catheter tube 1.

After installation of the medical access system on the patient, the needle device 39 is removed. The needle opening 25 is then used as a connecting option for, for example, an infusion line or an aspiration element, for example a syringe.

There may also be an injection port on the housing 20, which for example projects from the housing 20 on the side turned away from the fixing wings 23. The injection port is used to inject medications. It is otherwise sealed by means of a closure cap.

The needle device 39 furthermore has a closure element 34 with plugs, and a handling element 32 on the proximal end of the puncture needle 30. In the basic state, the needle opening 25 is sealed by the closure element 34. The handling element 32 is used for handling of the needle device 39 by the user, i.e. essentially for withdrawing the puncture needle 30 after puncture has been carried out. The needle device 39 is, as mentioned, removed after installation of the catheter 29 on the patient.

At the proximal end, i.e. in the region of the handling element 32, the needle device 39 may have an attachment port for attaching a syringe or an infusion line, for example for blood aspiration. The attachment port is sealed by a closure cap 40. The closure cap 40 may, for example, be screwed or plugged on.

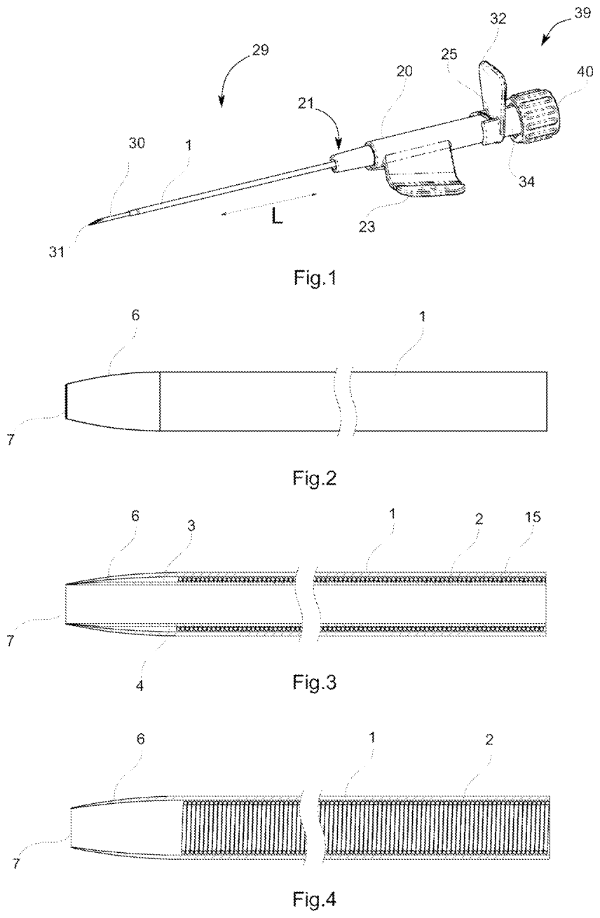

It may be seen in FIG. 2 that the catheter tube 1 is hose-shaped on the outer side, for example with a cylindrical outer shape. At the near-patient end, the catheter tube 1 ends with a dilation section 6 which tapers on the outer side toward the free end. At the free end, the catheter tube 1 has an axial opening 7 which is used as an outlet opening of the catheter tube 1 for the administration of medicaments. In the case of an indwelling cannula, the puncture needle 30 also extends from this opening 7.

FIG. 3 shows a cross-sectional view of a first embodiment of the medical access system, with a catheter tube 1 in which a spiral supporting structure 2 is arranged. As well as a tubular outer body 15, the catheter tube 1 in this case additionally has a tubular inner body 3 which is arranged inside the tubular outer body 15. An annular gap, in which the spiral supporting structure 2 is arranged with a certain radial play, is formed between the tubular outer body 15 and the tubular inner body 3. In a further advantageous embodiment, the spiral supporting structure 2 may be connected to the tubular inner body 3. The supporting structure 2 and the tubular inner body 3 then form a connected unit which is axially displaceable in relation to the tubular outer body 15.

It may also be seen that the tubular outer body 15 also tapers on the inner side toward the end in respect of the cross-sectional dimensions in the dilation section 6. The spiral supporting structure 2 is no longer arranged in this region, and there may be a free space 4 there.

FIG. 4 shows a further advantageous embodiment of a catheter tube 1 without the tubular inner body 3. It may be seen that the spiral supporting structure 2 is arranged directly inside the catheter tube 1 and is not covered toward the inner side. The puncture needle 30 is then guided directly inside the spiral supporting structure 2.

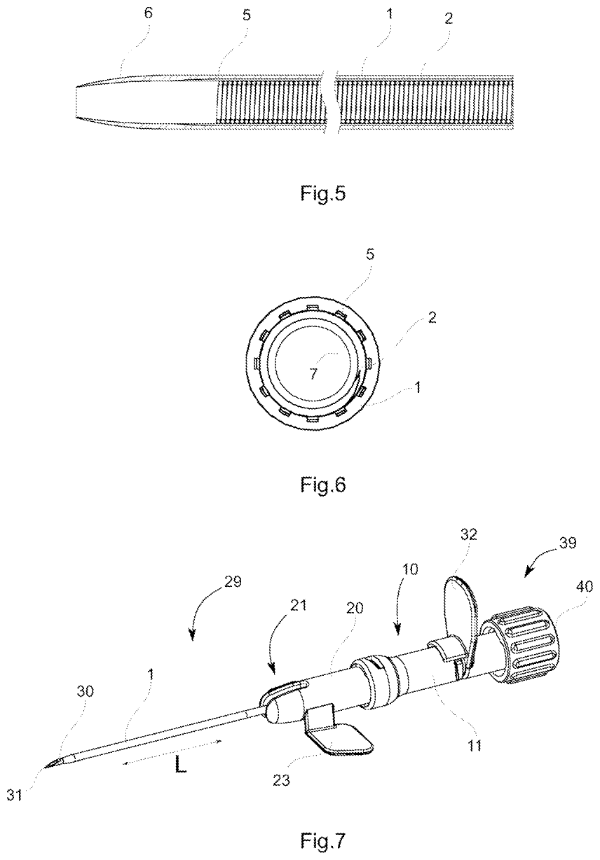

As a further advantageous embodiment, FIG. 5 shows a catheter tube 1 which is configured in a similar way to FIG. 4 but has a plurality of trench-like grooves 5 running in the longitudinal direction in a manner distributed over the circumference. The arrangement of the grooves 5 may be seen clearly in the cross-sectional representation of FIG. 6. The grooves 5 further reduce the friction between the catheter tube 1 and the spiral supporting structure 2. Such longitudinal grooves are also helpful for the visibility of a blood flow through the catheter tube 1.

As may be seen in particular from FIG. 6, the inner diameter of the opening 7 may be less than the outer diameter of the spiral supporting structure 2. Accordingly, the spiral supporting structure 2 cannot emerge from the opening 7.

The spiral supporting structure 2 may be formed for example from a metal wire or another suitable material. A round material or a flat material, for example with a rectangular or square cross section, may for example be used. The material of the spiral supporting structure 2 may be solid or internally hollow.

A further embodiment of a medical access system, in which the spiral supporting structure 2 can be withdrawn from the catheter tube 1, is explained with the aid of FIGS. 7 to 9. FIG. 7 shows the medical access system in the delivery state, i.e. all the components are in an assembled initial position. It may be seen that the medical access system has an additional module, specifically in the form of a supporting module 10, of which only a housing 11 of the supporting module 10 can be seen in FIG. 7. The housing 11 is coupled to the housing 20 of the catheter 29 using fastening elements. The handling element 32 of the needle device 39 is then located at the patient-remote end of the housing 11 of the supporting module 10.

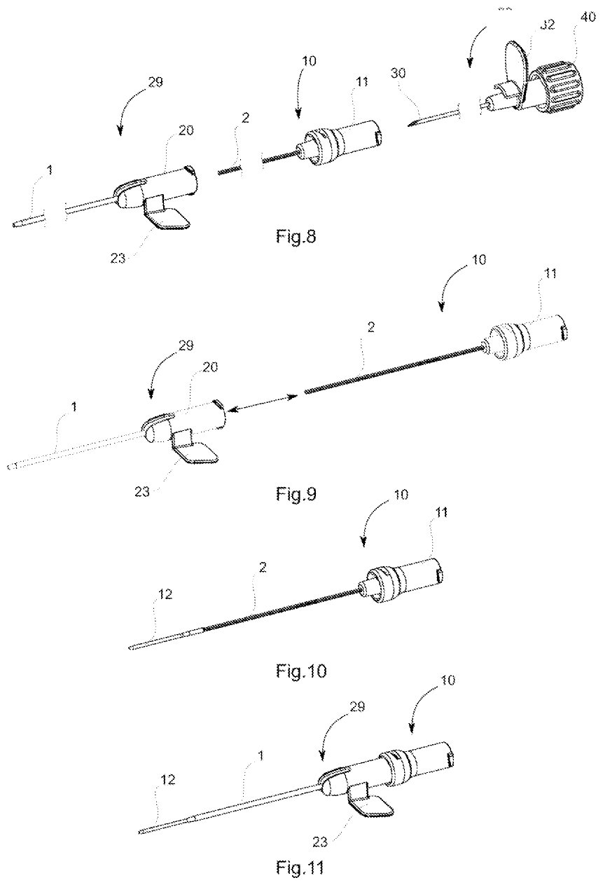

FIG. 8 shows the medical access system according to FIG. 7 in a kind of exploded representation, so that the individual modules can be seen. The following modules are therefore present: catheter 29, supporting module 10 and needle device 39. It may be seen that the spiral supporting structure 2 is secured to the housing 11 or another part of the supporting module 10. When the housing 11 is released from the housing 20, for example by a rotational movement, the housing 11 can be removed together with the spiral supporting structure 2 from the module constituted by the catheter 29. The needle device 39 may be removed from the supporting module 10 in a similar way.

FIG. 9 once more shows the complete supporting module 10 with the spiral supporting structure 2 after its removal from the catheter 29. Now, by way of example, an identically formed new supporting module 10 may be fastened to the catheter 29, i.e. a new spiral supporting structure 2 is pushed into the catheter 1.

Alternatively, a differently configured supporting module may also be applied to the catheter 29, for example an embodiment according to FIG. 10. In the embodiment of FIG. 10, an additional constituent part in the form of a tubular section 12 is applied to the near-patient end of the spiral supporting structure 2, for example by molding using an injection-molding method, welding, plugging and/or adhesive bonding. The spiral supporting structure 2 is lengthened at the near-patient end by this additional tubular section 12 to such an extent that, when it is plugged into the catheter tube 1, at least a part of the tubular section 12 protrudes therefrom as illustrated in FIG. 11. With such a tubular section 12 protruding from the catheter tube 1 at the near-patient end, a venous valve may for example be bridged.

In the case in which there is a tubular inner body 3 inside the spiral supporting structure 2, it may be configured to be lengthened at the near-patient end to such an extent that it forms such a tubular section 12 protruding from the catheter tube 1 at the near-patient end. The tubular inner body 3 then protrudes from the spiral supporting structure 2 by a substantial amount at the near-patient end.

The tubular inner body 3 or at least the tubular section 12 may for example be formed from plastic, optionally with addition of barium or inlaid markers consisting of another material in order to generate an improved radiopacity.

FIG. 11 shows the catheter 29 with the supporting module 10 according to FIG. 10 applied thereto. It may be seen that the tubular section 12 protrudes from the catheter tube 1.

### LIST OF REFERENCE SIGNS

1 catheter tube
2 spiral supporting structure
3 tubular inner body
4 free space
5 grooves
6 dilation section
7 axial opening
10 supporting module
11 housing of the supporting module
12 tubular section
15 tubular outer body
20 housing
21 tube exit region
23 fixing wing
25 needle opening
29 catheter
30 puncture needle
31 tip
32 handling element
34 closure element
39 needle device
40 closure cap
L longitudinal direction

1. A medical access system for providing an access to a hollow body of a patient, comprising:
at least one catheter with a vasiform catheter tube wherein the at least one catheter is designed to be
pushed over at least a part of a length of the catheter tube through an opening created by a puncture needle, or
pushed through a tunic of the hollow body after puncture of the tunic of the hollow body and to
wherein at least a part of the at least one catheter is designed to remain in the hollow body over a period of time,
wherein at least the part of the catheter tube designed to remain in the hollow body has at least one spiral supporting structure.

2. The medical access system as claimed in claim 1, wherein the at least one spiral supporting structure is arranged loosely in the catheter tube over an entire longitudinal extent or a predominant part of the longitudinal extent of the spiral supporting structure.

3. The medical access system as claimed in claim 1, wherein the at least one spiral supporting structure is secured permanently to the catheter tube or to a constituent part of the medical access system connected to the catheter tube using at least one fastening element.

4. The medical access system as claimed in claim 3, wherein the at least one spiral supporting structure has a first end and a second end, and wherein the at least one spiral supporting structure is secured at the first end, the second end, or at both the first end and the second end using the at least one fastening element and is otherwise arranged loosely in the catheter tube.

5. The medical access system as claimed in claim 1 wherein a bending moment of resistance and/or a radial moment of resistance of the catheter tube is increased by the at least one spiral supporting structure.

6. The medical access system as claimed in claim 1 further comprising a tubular inner body is arranged inside the at least one spiral supporting structure.

7. The medical access system as claimed in claim 1 wherein the at least one spiral supporting structure is secured releasably to the catheter tube or a constituent part of the medical access system connected to the catheter tube, and is selectively withdrawable from the catheter tube.

8. The medical access system as claimed in claim 1 further comprising a supporting module as a further module, present in addition to the at least one catheter, wherein the supporting module is coupled releasably to the at least one catheter, wherein the supporting module comprises the at least one spiral supporting structure.

9. The medical access system as claimed in claim 8, wherein the at least one spiral supporting structure is secured permanently to a part of the supporting module.

10. The medical access system as claimed in claim 1 wherein the at least one spiral supporting structure comprises a multiplicity of spiral turns which bear directly on one another.

11. The medical access system as claimed in claim 1 wherein the at least one spiral supporting structure comprises a multiplicity of spiral turnswhich loosely bear directly on one another but are not fastened to one another.

12. The medical access system as claimed in claim 1 wherein the at least one spiral supporting structure or any spiral turns of the at least one spiral supporting structure comprise a friction-reducing coating.

13. The medical access system as claimed in claim 1 wherein the at least one spiral supporting structure extends only over a part of a longitudinal extent of the catheter tube.

14. The medical access system as claimed in claim 1 wherein the catheter tube comprises a near-patient end with an axial opening, wherein an the inner diameter of the axial opening is less than an outer diameter of the at least one spiral supporting structure or at least an outer diameter of a near-patient end region of the at least one spiral supporting structure.

15. The medical access system as claimed in claim 1 wherein the catheter tube has a plurality of trench-like grooves which run linearly or in the manner of a coil in a longitudinal direction on an inner side of the catheter and in a manner distributed over a circumference of the catheter.

16. The medical access system as claimed in claim 1 further comprising a tubular section which protrudes at a near-patient end from the catheter tube through an axial opening formed at a free end of the catheter tube, wherein the tubular section projects at the near-patient end from the at least one spiral supporting structure.

17. The medical access system as claimed in claim 1 wherein the medical access system is configured as an indwelling cannula for puncturing a hollow body of a patient by a puncture needle wherein the indwelling cannula has at least the at least one catheter with the vasiform catheter tube, in which the puncture needle is guidable longitudinally displaceably, wherein the at least one catheter is designed to be pushed over at least a part of the length of the catheter tube through an opening created by the puncture needle through a tunic of the hollow body and wherein the indwelling cannula is designed to remain in the hollow body over a period of time.

18. The medical access system as claimed in claim 1 wherein the at least one spiral supporting structure consists predominantly or fully of one or more plastic materials.

19. The medical access system of claim 1 wherein the at least one spiral supporting structure is in a form of at least one spirally wound, internally hollow guide wire, and wherein the at least one spirally wound, internally hollow guide wire extends over an entire length of the at least one spiral supporting structure or over a predominant part of the entire length.
